(19) United States
(12) Patent Application Publication
(10) Pub. No.: US 2023/0190084 A1
(43) Pub. Date: Jun. 22, 2023

(54) Implantable Internal Observation Device and System

(71) Applicant: KARL STORZ Imaging, Inc., Goleta, CA (US)

(72) Inventors: Daniel Lietz, Santa Barbara, CA (US); Eric Hale, Vancouver, WA (US); Nathan J. Schara, Pasadena, CA (US); George E. Duckett, III, Castaic, CA (US)

(73) Assignee: KARL STORZ Imaging, Inc., Goleta, CA (US)

(21) Appl. No.: 17/553,506

(22) Filed: Dec. 16, 2021

(51) Int. Cl. A61B 1/05; A61B 1/06; A61B 1/045

(52) U.S. Cl. CPC A61B 1/05; A61B 1/045; A61B 1/0684

(57) ABSTRACT

A fully implantable observation instrument is presented enabling visualization of an internal region of a human or animal body without the need of wires extending from within the body to without. Data and power may be transmitted wirelessly from a communication assembly located near an accessible surface of the body, such as directly under the skin. The communication assembly is connected and delivers necessary power to an also implanted camera assembly that includes an electronic image sensor and a light source contained within an at least partially transparent biocompatible container.

### FIELD OF THE INVENTION

The present invention relates to an observation instruments that are implantable within a human or animal body, particularly one in the field of medical imagery and monitoring, as well as deployable observation instruments for industrial purposes.

### BACKGROUND OF THE INVENTION

Endoscopic examination techniques have prevailed in a multiplicity of medical and veterinary fields of application, as well as in many non-medical fields. In such examination techniques, an endoscope, which has an elongate shaft with an imaging optical system, is introduced into an internal cavity of a human or animal body or another object to be examined. Endoscopes are generally introduced into the body either through a natural bodily orifice, such as the mouth, or through a small incision that gives access to the observation site. Minimally invasive surgeries are thereby enabled, by the insertion of observational endoscopes as well as operational tools through one or more small incisions. These standard methods of observation of an internal area of the human body generally require constant attention and are only employed during the actual medical intervention, such as a surgery or the passage of an endoscopic capsule through the digestive system.

Traditional endoscopic monitoring solutions are clearly limited when long term monitoring of an internal area of the body is desired. In addition, endoscopes and endoscopic capsules each have their own associated drawbacks, expertise and some degree of invasive surgery for the endoscope, and hazardous materials, including batteries, being introduced into the body with endoscopic capsules. Additionally, there are limitations on regions of the body where these systems may be deployed.

In order to monitor internal sites in the longer term, some wired implantable endoscopic devices have also been suggested, such as in U.S. Pat. Publication No. 2014/0046132 A1 (Filed Feb. 13, 2014), hereby incorporated by reference. However, prior art systems, such as these come with their own limitations, including requiring an interface between the internal camera system and an external power supply and/or relay as well as an accompanying potential for infection, discomfort, inconvenience for the patient, and potential accidental damage to the system or it’s wiring.

Additionally, there are various non-invasive scan procedures that can be performed, such as X-rays, CAT and PET scans, as well as ultrasonic imaging. However, these procedures can be quite expensive, require additional equipment, and do not allow for direct optical visualization of the desired area. Additionally, when frequent repeated imaging is necessary, some of these techniques are not optimal, due to the preparation necessary and exposure, in some cases, to very low wavelength radiation.

What is needed is a long-term, internal monitoring device that minimizes the introduction of toxic substances into the body and exposure of radiation thereto. Additionally the device or system should ideally limit the likelihood of infection while being relatively convenient to the patient and not prone to accidental damage.

### BRIEF DESCRIPTION OF THE INVENTION

An implantable observation device without wires extending from inside of the body to outside of the body, and system capable of the long-term observation of a site of interest is provided. Most embodiments presented herewith are directed at implantation within a living human or animal body, however, certain implementations may also be useful for industrial applications.

### BRIEF DESCRIPTION OF THE DRAWINGS

Further aspects of the present invention will be apparent from the figures and from the description of embodiments that follow. The figures are given by way of illustration only, and thus are not limitative of the present invention. The index numbers used throughout attempt to convey uniformity as much as possible, while also permitting distinct reference thereto. Therefore, the numbering system employed is for the sake of simplicity and clarity and should not be considered limiting.

FIG. 1 shows the basic constituents of an embodiment of an implantable observation device and corresponding communication element.

FIG. 2 illustrates the observation device implanted within a body as an element of an observation system further incorporating a data receiving element and charging element.

For clarity not all reference numerals are displayed in all figures. If a reference numeral is not explicitly mentioned in the description of a figure, it has the same meaning as in the other figures.

### DETAILED DESCRIPTION OF THE INVENTION

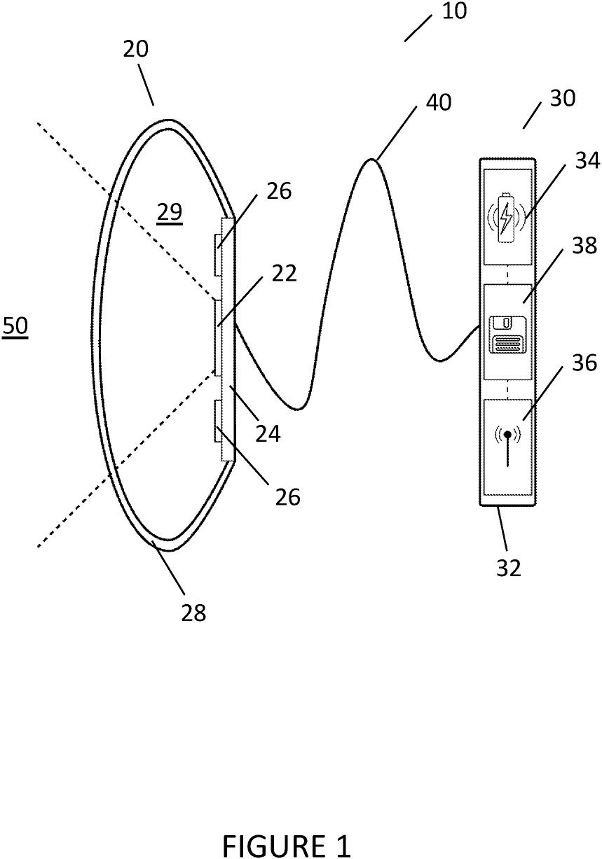

The various elements of an example embodiment of the implantable observation instrument are shown in FIG. 1. Observation device 10 includes a camera assembly 20 and a communication assembly 30 connected by an electronic communication cable 30. This communication cable 40 is capable of transferring image data from the camera assembly 20 to the communication assembly 30 as well as supply the camera assembly with electrical power and issue control commands to, or directly control, the various elements of the camera assembly from the communication assembly.

The camera assembly 20 may include a primary mounting/circuit board 24 with all necessary electrical connections to communicate with devices attached thereto. Mounted upon the circuit board is an image sensor 22 such as a CMOS or CCD camera and any associated optical elements comprising an objective lens necessary to form an image of a scene 50 on the image plane of the image sensor 22. Also attached to the mounting board 24 are one or more illumination sources 26, such as light emitting diodes (LEDs), to provide illumination to the subject scene 50. Attached to the mounting board 24 is a camera container 28. This container is made of a biocompatible material, and is transparent at least in the region of the field of view of the image sensor 22 as indicated by dotted lines in FIG. 1, such that both illumination light from the illumination source or sources 26, such as LEDs as well as light reflected or scattered by the subject scene 50 may pass therethrough. Thereby the scene 50 is illuminated and light collected by the camera assembly 20. The mounting/circuit board may consist of multiple layers, including an exterior layer made of biologically inert/biocompatible materials if exposed to biological matter, as is illustrated in the embodiment of FIG. 1. Alternate embodiments may include the entire mounting/circuit board 24 enclosed by the camera container 28. Further the shape of the mounting board may be specifically selected such that it provides support for its positioning within the body. As shown in the embodiment of FIG. 1, the positioning of the mounting board 24 provides the camera assembly 20 with one flat side, which may be useful in preventing the device from moving within its selected location within the body. Other shapes of the board 24, or indeed, of the entire unit 20 are possible.

Within the camera container 28 is an internal device space 29. This region may be occupied by any material appropriate to the situation. For example, in some embodiments, the internal device space 29 may be a solid mass of optically transparent material, such as used for the transparent regions of the camera container 28 and may be a single unit therewith molded directly around and over the primary mounting/circuit board 24. In other embodiments the internal device space 29 may be filled with an appropriate, optically transparent liquid or gas. Pressure within the internal space 29 on the camera container 28 may define the shape of the camera assembly 20. In some embodiments, the camera assembly may be inserted into its observation location, such as in a body lumen, in a “deflated” state. This can minimize the size of the incision necessary for its insertion into a human or animal body, and then can be “inflated” by the introduction of an appropriate fluid into the internal device space, such as by injection of air by a syringe or other appropriate means. The camera assembly could include an appropriate port for such an injection or could be include a self-sealing surface. When the camera assembly 20 is later to be removed, the material within the internal device space could be removed by a similar manner, or, if appropriate, be allowed to vent into the body by piercing the camera container 28, decreasing its size, thereby, and facilitating its removal. It should also be noted that the shape of the camera container may be specifically selected in order to provide optimal observational characteristics. For example, in some embodiments the index of refraction of any material within the internal device space 29 and that of the camera container 28 could be selected and matched to the final shape of the camera assembly, such that its optical properties behoove the optical system of the image sensor 22. Certain configurations of materials and shapes, for example, could supply a wide-angle lensing effect, minimizing other optical elements necessary the objective system of the image sensor 22. Additionally, the shape of the camera assembly 20, including the camera container 28 and the internal device space 29, will generally be selected such that adequate distance between the desired image scene 50 and the image sensor 22 is present, such that a minimum necessary focus distance, at least, is maintained.

Connected to the camera assembly 20 by an electronic communication cable 40 is the communication assembly. Within an outer communication container 32, also made of a biocompatible material, are housed necessary means for powering the camera assembly 20 as well as for communications outside the body. A power supply element 34 may include a battery element that may be remotely chargeable by means known in the art, such as by inductive charging and Qi charging methods. Such methods allow the charging of a battery or supplying of power inductively at a short distance without the need for direct electrical connections. By this means a battery element within the power supply 34 may be charged or power may be directly supplied inductively through the power supply element 34 to the camera assembly 20. For example, in some implementations of the invention, constant monitoring may be desired, whereby power supply element would include a battery or super capacitance element that could be externally charged (or charged prior to implantation), and thereby the camera assembly could collect images of the object scene 50 over time (either continuously or at discrete intervals). Alternately some implementations of the observation device 10 may be primarily intended for direct, real-time viewing, such as at regularly scheduled check-ups. In such implementations a patient would return to a practitioner, and at that time, an external supply of inductive energy could be applied to the power supply, and a live video feed be observed. In this way potentially hazardous materials introduced into the body could be avoided by the non-inclusion of a chemical battery element within the power supply 34.

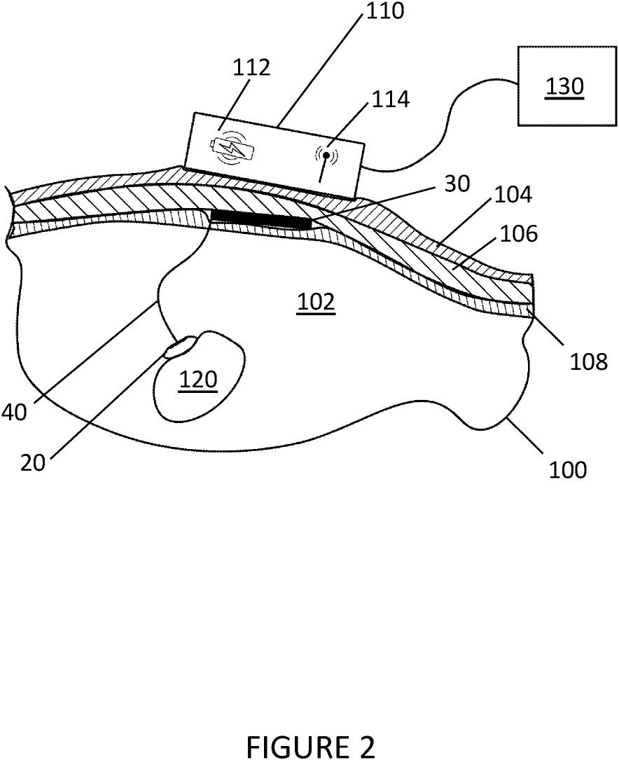

Another element of the communication assembly 30 is a first wireless data transmitter/receiver 36. This element can include any known, appropriately sized and powered short-range, wireless communication devices with such communications modes such as UWB, Wi-Fi, ZigBee and bluetooth. The first wireless data transmitter/receiver 36 can be powered directly by the power supply 34, and will communicate, as will be described further with reference to FIG. 2, with a wireless data receiving unit. It should be noted that the first wireless data transmitter/receiver 36, while primarily used for transmitting wireless data may also be capable of receiving wireless commands, updates to any on board memory or firmware, etc.

The communication assembly 30 may also optionally include a memory storage element 38 where image data transmitted from the camera assembly 20 may be stored as well as any data storage necessary to control the observation device, system firmware, etc. The memory storage device may comprise any appropriate storage device known in the art, such as SIM cards, FPGAs, EPROMs, etc., and the type of storage may be selected depending on the specific needs of a particular observation device 10. For example, an observation device intended for use only at regularly scheduled doctor visits may not need long term storage capacity, and therefore any storage element 38 may be limited to necessary system control storage.

In preferred embodiments of the invention, the size and shape of the communication container 32 will be selected to be both minimally invasive, as well as positional integrity, such that they will be easily detectable when not directly visible (such as when implanted under the skin), and not liable to move once installed in their desired locations. This, in most preferred embodiments, the communication assembly will be as small and non-intrusive as possible, so as to not overly inconvenience the patient while simultaneously being accessible when necessary to interact with the observation device.

FIG. 2 shows an observational device 10 implanted within a body 100. The body 100 is presented only as a simplified example, in order to describe the various elements of the invention. The camera assembly 20 is positioned within the internal body space 102 and, if appropriate, inflated, such that it will collect image data from an object of interest 120, such as an organ, an open body lumen, an area where surgery was previously performed, a potential tumor, etc. The camera assembly 20 is connected by communication cable 40 to the communication assembly 30, which is in turn placed, in this example, beneath an epidermal layer 104 and a fat layer 106, above a muscle layer 108. This positioning makes the communication assembly 30 accessible via wireless communication to regions exterior to the body 100.

A receiving assembly 110 can then be placed in the vicinity of the communication assembly 30, and usually in contact with the epidermal layer 104 of the body 100. The receiving assembly 110 contains a wireless power transmission system 112, such as an inductive power supply such as the Qi specification. This power transmission system 112 provides electrical power to the power supply 34 of the communication assembly, powering and/or charging the implanted observation device. The receiving assembly also includes a second wireless data transmitter/receiver, which can communicate wirelessly with the first wireless data transmitter/receiver 36 of the communication assembly 30, downloading, thereby any image data collected by the camera assembly 20 and/or stored in the data storage 38.

Received image data can then be transmitted to a connected camera control unit 130, which can, in turn, display, store, analyze, etc., the received image data. After the intervention is complete, for example, when the battery is fully charged, the image data is transferred, and/or the live observation is complete, the patient generally will require no further invasive procedure, and the observation device may be left within the body 100 or removed at such a time as it is no longer necessary.

Additionally, the camera control unit 130 may issue commands through the receiving assembly 110 and its second wireless data transmitter/receiver 114 to the first wireless data transmitter/receiver 36 of the communication assembly 30 in order to update any on board firmware, issue any memory commands, such as to erase the stored memory, turn on and off the LEDs, set timers for image acquisition, etc.

While the invention has been described in connection with various embodiments, it will be understood that the invention is capable of further modifications. This application is intended to cover any variations, uses, or adaptations of the invention following, in general, the principles of the invention, and including such departures from the present disclosure as, within the known and customary practice within the art to which the invention pertains.

Reference numerals

| | |
|---|---|
| 10 | Observation device |
| 20 | Camera assembly |
| 22 | Image Sensor with objective |
| 24 | Circuit board |
| 26 | Illumination source |
| 28 | Camera container |
| 29 | Internal device space |
| 30 | Communication assembly |
| 32 | Communication container |
| 34 | Power supply |
| 36 | First wireless data transmitter/receiver |
| 38 | Storage |
| 40 | Electronic communication cable |
| 50 | Image scene |
| 100 | Body |
| 102 | Internal body space |
| 104 | Epidermal layer |
| 106 | Fat layer |
| 108 | Muscle layer |
| 110 | Receiving assembly |
| 112 | Wireless power transmitter |
| 114 | Second wireless data transmitter/receiver |
| 120 | Object of interest |
| 130 | Camera control unit |

1. An implantable internal observation device comprising
a camera assembly comprising an image sensor, an illumination source, a biocompatible camera container containing the image sensor and illumination source therein, the biocompatible camera container being optically transparent at least in a region within a field of view of the image sensor;
a communication assembly comprising a power supply and a first wireless data transmitter/receiver contained within a biocompatible communication container, and where the communication assembly is connected to the camera assembly.

2. The implantable internal observation device of claim 1, further comprising an electronic communication cable connecting the camera assembly to the communication assembly.

3. The implantable internal observation device of claim 1, wherein the power supply comprises a battery.

4. The implantable internal observation device of claim 1, wherein the power supply does not include a battery.

5. The implantable internal observation device of claim 1, wherein the power supply comprises an inductive power transfer means.

6. The implantable internal observation device of claim 1, further comprising memory storage element configured to store images captured by the image sensor, and wherein the power supply comprises a battery.

7. The implantable internal observation device of claim 1, wherein an internal device space within the biocompatible camera container is filled with a transparent fluid.

8. The implantable internal observation device of claim 7, further comprising a port wherein the fluid may be introduced into the camera container.

9. The implantable internal observation device of claim 1, wherein the illumination source comprises a plurality of light emitting diodes.

10. An observation system comprising
an implantable observation device comprising a camera assembly comprising an image sensor, an illumination source, a biocompatible camera container containing the image sensor and illumination source therein, the biocompatible camera container being optically transparent at least in a region within a field of view of the image sensor, and a communication assembly comprising a power supply and a first wireless data transmitter/receiver contained within a biocompatible communication container, and where the communication assembly is connected to the camera assembly;
a receiving assembly comprising a wireless power transmitter and a second wireless data transmitter/receiver; and
a camera control unit in communication with the receiving assembly.

11. The observation system of claim 10, wherein the communication assembly further comprises a memory storage element.

12. A method for collecting image data from a region within a human or animal body comprising the steps of
inserting into a human or animal body an implantable observation device, the implantable observation device comprising a camera assembly comprising an image sensor, an illumination source, a biocompatible camera container containing the image sensor and illumination source therein, the biocompatible camera container being optically transparent at least in a region within a field of view of the image sensor;
directing a field of view of the camera assembly toward a region of interest within the body;
inserting into the body near an accessible region of the body, a communication assembly comprising a power supply and a first wireless data transmitter/receiver contained within a biocompatible communication container, where the communication assembly is connected to the camera assembly;
placing, within the vicinity, but outside of the body, a receiving assembly comprising a wireless power transmitter and a second wireless data transmitter/receiver; and transferring image data, wirelessly, from the first wireless data transmitter/receiver to the second wireless data transmitter/receiver.

13. The method of claim 12 comprising the further step of transmitting, wirelessly, power from the wireless power transmitter to the power supply.

14. The method of claim 12 comprising the further step of illuminating the region of interest with the illumination source.

15. The method of claim 14, wherein the step of transmitting image data comprises the step of receiving image data collected in real time by the image sensor.
